(19) United States
(12) Patent Application Publication
(10) Pub. No.: US 2013/0267827 A1
(43) Pub. Date: Oct. 10, 2013

(54) METHOD AND MAGNETIC RESONANCE SYSTEM FOR FUNCTIONAL MR IMAGING OF A PREDETERMINED VOLUME SEGMENT OF THE BRAIN OF A LIVING EXAMINATION SUBJECT

(71) Applicants: David Grodzki, Erlangen (DE); Bjoern Heismann, Erlangen (DE); Jeanette Lenger, Wuerzburg (DE); Sebastian Schmidt, Weisendorf (DE)

(72) Inventors: David Grodzki, Erlangen (DE); Bjoern Heismann, Erlangen (DE); Jeanette Lenger, Wuerzburg (DE); Sebastian Schmidt, Weisendorf (DE)

(21) Appl. No.: 13/857,322

(22) Filed: Apr. 5, 2013

(30) Foreign Application Priority Data
Apr. 5, 2012 (DE) 102012205629.7

(51) Int. Cl. A61B 5/04; A61B 5/0476; A61B 5/00

(52) U.S. Cl. CPC A61B 5/04012; A61B 5/0042; A61B 5/0476
USPC 600/411

(57) ABSTRACT

In a method and a magnetic resonance (MR) system for functional MR imaging of a predetermined volume segment of the brain of a living examination subject, MR data of the predetermined volume segment are acquired, EEG data of the examination subject are acquired with the acquisition of the EEG data taking place simultaneously with the acquisition of the MR data, and the MR data automatically evaluated dependent on the acquired EEG data.

### BACKGROUND OF THE INVENTION

1. Field of the Invention

The present invention concerns a method and a magnetic resonance (MR) system for functional MR imaging (fMRI) in which MR exposures of the brain of a living examination subject (in particular a person) are generated.

2. Description of the Prior Art

“Resting state fMRI” is an MR method in which MR exposures of a patient at rest are created. As in classical fMRI, in these MR exposures a signal change is determined by means of the BOLD (“Blood Oxygen Level Dependent”) effect, which represents a measure of the physiological activation of specific areas of the brain.

In contrast to classical fMRI in which the patient is exposed to specific stimuli, or in which specific tasks are posed to the patient, in resting state fMRI the MR exposures are created with the patient at rest. A time correlation of the activation of specific brain centers thereby appears, this correlation being determined by a degree of interlinking of these centers. Relevant diagnostic information (about psychiatric illnesses, for example) can in turn be obtained.

The MR measurements in combination with morphological MR acquisitions for resting state fMRI can take 15 minutes or longer. The risk thus exists that the activation state of the patient can change (for example since the patient falls asleep), which negatively leads to irrelevant activation patterns and adulterates the results or even emulates false diagnoses.

### SUMMARY OF THE INVENTION

An object of the present invention is to at least reduce these problems according to the prior art.

According to the invention, a method is provided for functional MR imaging of a predetermined volume segment of a brain of a living examination subject. The method includes the steps of acquiring MR data of the predetermined volume segment, acquiring EEG data of the examination subject, with the acquisition of the EEG data and the acquisition of the MR data taking place simultaneously, and the MR data are evaluated depending on the acquired EEG data.

By the simultaneous acquisition of the MR data and the EEG data it is possible to check (using the EEG data) whether the desired activation state of the patient is present during the acquisition of the MR data. It is thus possible to evaluate the MR data depending on the respective activation state established by the EEG data, or to only evaluate MR data that were acquired during the desired activation state of the patient.

A spectral analysis of the EEG data can be implemented, for example by generating the frequency spectrum of the acquired EEG data. The evaluation of the MR data can then take place depending on the spectral analysis or depending on the detected frequency spectrum.

The current activation state of the patient can be determined using the spectral analysis or using the frequency spectrum. Since the evaluation of the MR data takes place depending on the spectral analysis or depending on the detected frequency spectrum, for example, only the MR data can then be evaluated that were acquired while the patient exhibited a desired activation state.

In a preferred embodiment according to the invention, the simultaneous acquisition of the MR data and the EEG data takes place in multiple successive time intervals or time slices. A frequency spectrum of the EEG data acquired during this time slice is thereby determined for each of these time slices. Depending on the frequency spectrum determined for the respective time slice, a class is determined for the respective time slice. The MR data acquired during the respective time slice are then also associated with this class, such that the MR data acquired during the multiple time slices are associated with different classes. To evaluate the MR data, the MR data of a specific class are evaluated depending on this class so that the MR data of one specific class are evaluated differently than the MR data of another specific class.

According to a first variant of this preferred embodiment, the frequency spectrum of the EEG data is subdivided into a predetermined number of frequency bands. One example of this subdivision is a subdivision of the frequency band into delta waves, theta waves, alpha waves, beta waves and gamma waves. The number of classes corresponds to the number of frequency bands, such that each class corresponds to one of these frequency bands. According to this first variant, it is determined in which of these frequency bands the EEG data are predominantly situated. The class corresponding to this frequency band is then also the class of the respective time slice so that the MR data that were acquired during this time slice are associated with this class.

In other words: for each time slice it is determined in which frequency band or in which frequency class is situated the largest proportion of the frequency spectrum of the EEG waves that were acquired during this time slice. The MR data acquired during this time slice are then also associated with this frequency class. A number of data sets of MR data then exist at the end of the MR measurement according to the invention, wherein the number of these data sets of MR data corresponds to the number of frequency bands or frequency classes insofar as MR data were acquired for each frequency class (meaning that the number of data sets of a frequency class or class can also be zero).

For example, if one of the classes (frequency classes) corresponds to the classical a wave frequency class, at the end of the method according to the invention a data set exists that is composed of MR data that were acquired in those time slices during which the EEG data or EEG waves of the examination subject predominantly corresponded to a waves. It is thereby possible to use only the MR data of this alpha wave frequency class for evaluation, and to discard the other MR data.

It is thereby possible to evaluate only those MR data that were acquired in a time period during which the examination subject exhibited a predetermined, desired activation state. An adulteration of the MR data by the acquisition of MR data during an unwanted activation state thus can be nearly precluded.

According to a second variant of the preferred embodiment, the frequency spectrum of the EEG data is also subdivided into a predetermined number of frequency bands. This subdivision can again correspond to the classical subdivision into the frequency bands or frequency classes alpha, beta, gamma, delta, theta. As in the first variant, in the second variant a number of predetermined classes also exists, but the number of predetermined classes does not need to correspond to the number of frequency classes in the second variant. In the second variant, each predetermined class is defined by frequency proportions of the EEG data within the defined frequency bands. In other words: each predetermined class is defined by a frequency proportion within the first frequency band, a frequency proportion within the second frequency band, . . . , and a frequency proportion within the last of the predefined frequency bands. In order to now assign the EEG data acquired within a defined time slice to one of these predetermined classes, the frequency proportions of the acquired EEG data within the predetermined frequency bands are determined. The class of the time slice then corresponds to that of the predetermined classes in which the predefined frequency proportions best correspond to the frequency proportions of the acquired EEG data.

In order to determine this, for example, a desired value can be determined for each of these predetermined classes for each of the defined frequency bands. The differences between the frequency proportion of the detected EEG waves in this frequency band and the desired value of this frequency band of this class can then be determined for each class for each frequency band. The respective time slice is associated with that class in which these differences are smallest. For example, for each predetermined class the sum of the absolute values of the differences between the frequency proportion of the detected EEG waves in the respective frequency band and the desired value of this class can be determined for this frequency band. The predetermined class in which this sum is smallest is then associated as a class to the respective time slice.

In this second variant, EEG data (and therefore the MR data) of a time slice can be divided according to schemes that are more complicated in comparison to the first variant. More complicated activation states (for example an activation state caused by visual stimulus, an activation state caused by auditory stimulus, or an activation state in which no external stimulus is present (resting state)) can be differentiated by the evaluation of the EEG data, and the acquired MR data can be subdivided into corresponding classes. For example, if only the MR data which were acquired during a “resting state” activation state are to be evaluated, the activity of different function networks in the brain in this “resting state” activation state can be detected and depicted. In other words: the MR data acquired in different activation states can be evaluated separately in order to separately detect the activity of different function networks (each activation state has its own function network).

The evaluation of the MR data can be the generation of morphological MR images in which active brain centers of the examination subject are detectable as such during the acquisition of the MR data.

According to a further embodiment of the invention, the MR data and the EEG data are acquired in multiple successive time intervals. For each time interval a decision is made as to whether the frequency spectrum of the EEG data acquired in this time interval is situated predominantly in a desired frequency band that was previously established. Only if this is the case are the MR data of the corresponding time interval evaluated; otherwise, these MR data are discarded. Only if the sum of time intervals in which the MR data of the evaluation were supplied (meaning that the frequency spectrum of the EEG data acquired in this time interval was predominantly situated in the desired frequency band) is larger than a predetermined time interval does the method end.

This embodiment guarantees that, overall, MR data corresponding to the duration of the predetermined time interval are acquired, wherein during the acquisition of these MR data the examination subject has a desired activation state that is characterized by the frequency spectrum of the acquired EEG data.

According to the invention it is also possible for user information to be provided is an output depending on the frequency spectrum of the acquired EEG data.

The operator of the magnetic resonance system thus can be warned, for example, in the event that no evaluable MR data could be generated or acquired over a determined time period. For example, the operator of the magnetic resonance system could be warned when no alpha waves of the examination subject are detected for the duration of a defined time period, which means that, in the defined time period, there is no time slice in which the frequency proportion of the EEG data is predominantly in the alpha frequency band.

According to the invention, the examination subject or the patient can also be directly informed by means of the user interaction. For example, user information could be generated when a predetermined time interval has predominantly delta waves along the frequency spectrum of the acquired EEG waves, which indicates that the patient has fallen asleep. In this case, the user information can be used to play a noise via headphones worn by the patient, in order to wake the patient. In contrast to this, if predominantly gamma waves are established in the frequency spectrum of the acquired EEG waves, the patient could be asked to relax by the user information. The opening or closing of the eyes can also be instigated by user information if the frequency spectrum of the acquired EEG waves lies predominantly in the alpha or beta frequency band.

According to another embodiment according to the invention, the EEG data of a specific time period are lowpass-filtered, such that only EEG data at a frequency below a frequency threshold are let through the corresponding lowpass filter. If the proportion of lowpass-filtered EEG data (i.e. the proportion of EEG data at frequencies below the frequency threshold) is above a predetermined proportion threshold, the MR data of this time period are discarded. In this case (when the proportion of the lowpass-filtered EEG data is above the predetermined proportion threshold) it is thereby possible to wake the patient since the patient has probably fallen asleep.

With this very simple embodiment according to the invention, the MR data from time periods in which predominantly delta or theta waves (i.e. EEG data with a frequency below 8 Hz) are present are advantageously eliminated from the MR data that are ultimately to be evaluated. Moreover, higher frequency interference due to the magnetic resonance system is advantageously prevented by the lowpass-filtering.

The present invention also encompasses a magnetic resonance system to generate an MR image of an examination subject. The magnetic resonance system has a basic field magnet, a gradient field system, at least one RF transmission antenna, at least one reception coil element, a control device, and an electroencephalograph. The control device serves to control the gradient field system and the at least one RF transmission antenna. Moreover, the control device is designed in order to receive measurement signals which have been acquired by the at least one reception coil element, and to evaluate these acquired measurement signals and create corresponding MR data. The magnetic resonance system is designed to acquire EEG data by means of the electroencephalograph simultaneously with the MR data. The control device then evaluates the MR data depending on the simultaneously acquired EEG data

The advantages of the magnetic resonance system according to the invention essentially correspond to the advantages of the method according to the invention described above.

The present invention also encompasses an non-transitory, computer-readable data storage medium that can be loaded into a memory of a programmable control device or computer of a magnetic resonance system. All or some embodiments of the method according to the invention that are described above can be executed when the control device executes the programming instructions. The programming instructions may make use of standard items such as libraries and auxiliary functions in order to realize the embodiments of the method. The programming instructions can be in source code (C++, for example) that must still be compiled and linked or that only needs to be interpreted, or can be an executable software code that has only to be loaded into the computer or control device for execution.

The electronically readable data medium can be, for example, a DVD, a magnetic tape or a USB stick on which is stored electronically readable control information.

In comparison to the prior art, the present invention offers a more robust and simpler examination of the brain by means of a magnetic resonance system.

The present invention is particularly suitable for “resting state” fMRI methods, but is not limited to this preferred field of application, since the present invention can also be used for fMRI methods in which activation states other than resting state are specifically presented or examined.

### BRIEF DESCRIPTION OF THE DRAWINGS

FIG. 1 schematically illustrates a magnetic resonance system according to the invention.

FIG. 2 shows an example of six classes of EEG data which are defined by specific frequency proportions in specific frequency bands.

FIG. 3 depicts a division of the EEG data acquired within a time slice into predetermined classes.

FIG. 4 is a flowchart of an embodiment of the method according to the invention.

### DESCRIPTION OF THE PREFERRED EMBODIMENTS

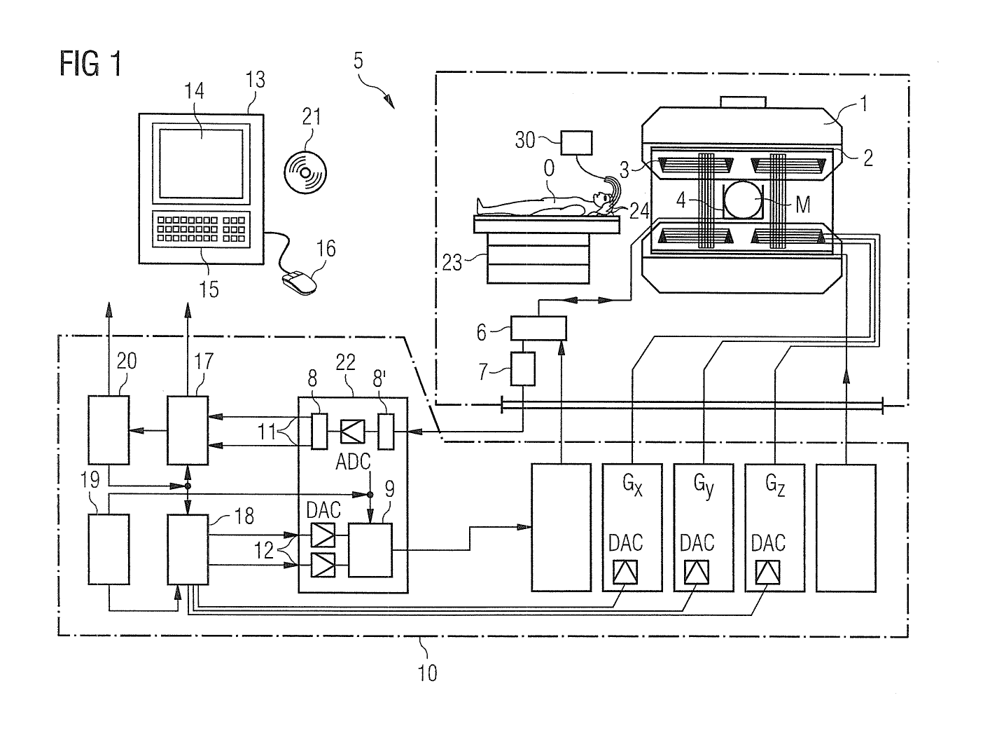

FIG. 1 is a schematic illustration of a magnetic resonance system 5 (a magnetic resonance imaging or magnetic resonance tomography apparatus). A basic field magnet 1 generates a temporally constant, strong magnetic field for polarization or alignment of the nuclear spins in a volume segment of a subject O, for example of a part of a human body that is to be examined. The subject O lying on a table 23 is driven into the magnetic resonance system 5 for examination or measurement (data acquisition). The high homogeneity of the basic magnetic field that is required for the magnetic resonance measurement is defined in a typically spherical measurement volume M into which the parts of the human body that are to be examined are introduced. Shim plates made of ferromagnetic material are attached at suitable points to support the homogeneity requirements, and in particular to eliminate temporally invariable influences. Temporally variable influences are eliminated by shim coils 2. The magnetic resonance system 5 that is shown also has an electroencephalograph 30 with which EEG data of the brain of the examination subject O are acquired simultaneously with the MR data. The EEG data are acquired at specific measurement points at the head of the patient.

A cylindrical gradient coil system 3 that has three sub-windings is inserted into the basic field magnet 1. Each sub-winding is supplied with current by an amplifier so as to generate a linear (also temporally variable) gradient field in a respective direction of the Cartesian coordinate system. The first sub-winding of the gradient field system 3 generates a gradient Gₓ in the x-direction; the second sub-winding generates a gradient G_{y} in the y-direction; and the third sub-winding generates a gradient G_{z} in the z-direction. Each amplifier has a digital/analog converter that is activated by a sequence controller 18 for accurately-timed generation of gradient pulses.

Located within the gradient field system 3 are at least one or more radio-frequency antennas 4 that convert the radio-frequency pulses emitted by a radio-frequency power amplifier into an alternating magnetic field for excitation of the nuclei and tipping the nuclear spins of the subject O to be examined or of the region of the subject O that is to be examined out of alignment with the basic field. Each radio-frequency antenna 4 has of one or more RF transmission coils and multiple RF reception coil elements in the form of an annular, or linear or matrix-like arrangement of component coils. The alternating field emanating from the precessing nuclear spins—normally the nuclear spin echo signals caused by a pulse sequence composed of one or more radio-frequency pulses and one or more gradient pulses—is also converted by the RF reception coil elements into a voltage (measurement signal) that is supplied via an amplifier 7 to a radio-frequency reception channel 8 of a radio-frequency system 22. The radio-frequency system 22 furthermore has a transmission channel 9 in which the radio-frequency pulses are generated for the excitation of the nuclear magnetic resonance. The respective radio-frequency pulses are digitally represented in the sequence controller 18 as a series of complex numbers based on a pulse sequence predetermined by the system computer 20. This number sequence is supplied as a real part and imaginary part to a digital/analog converter (DAC) in the radio-frequency system 22 via respective inputs 12, and from the digital/analog converter to the transmission channel 9. In the transmission channel 9 the pulse sequences are modulated on a radio-frequency carrier signal whose base frequency corresponds to the center frequency.

Switching from transmission operation to reception operation takes place via a transmission/reception diplexer 6. The RF transmission coil of the radio-frequency antenna 4 radiates the radio-frequency pulses for excitation of the nuclear spins into the measurement volume M and detects resulting echo signals via the RF reception coils. The acquired magnetic resonance signals are phase-sensitively demodulated to an intermediate frequency in a reception channel 8′ (first demodulator) of the radio-frequency system 22 and digitized in an analog/digital converter (ADC). This signal is further demodulated to a frequency of zero. The demodulation to a frequency of zero and the separation into real part and imaginary part occur in a second demodulator 8 after the digitization in the digital domain. An MR image or three-dimensional image data set is reconstructed by the image computer 17 from the measurement data acquired in such a manner. The administration of the measurement data, the image data and the control programs takes place via the system computer 20. Based on a specification with control programs, the sequence controller 18 monitors the generation of the respective desired pulse sequences and the corresponding scanning of k-space. In particular, the sequence controller 18 thereby controls accurately-timed activation of the gradients, the emission of the radio-frequency pulses with defined phase amplitude and the reception of the nuclear magnetic resonance signals. The time base for the radio-frequency system 22 and the sequence controller 18 is provided by a synthesizer 19. The selection of corresponding control programs to generate an MR image (which control program are stored on a DVD 21, for example) and the presentation of the generated MR image take place via a terminal 13 which comprises a keyboard 15, a mouse 16 and a monitor 14.

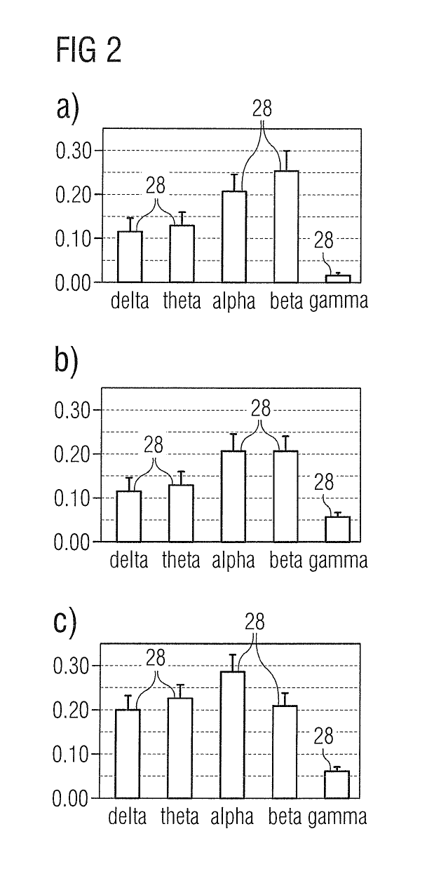

Six predetermined classes of EEG data are shown in FIGS. 2a through 2f. Each of these six classes is defined by five frequency proportions 28, wherein each frequency proportion 28 indicates what proportion of the frequency spectrum of the EEG data lies within the corresponding classical frequency band or frequency class. The classical frequency bands are the delta waves in a frequency range from 0.1 to 4 Hz; the theta waves in a frequency range from 4 to 8 Hz; the alpha waves in a frequency range from 8 to 13 Hz; the beta waves in a frequency range from 13 to 30 Hz; and the gamma waves in a frequency range above 30 Hz.

Shown in FIG. 2a is a class of EEG data that are generated by a healthy brain when the associated patient is not exposed to any stimuli, which is also known as default mode or resting state. It is apparent that, in the default mode class, the frequency proportion of the delta waves is approximately 12%; the frequency proportion of the theta waves is approximately 13%; the frequency proportion of the alpha waves is approximately 21%; the frequency proportion of the beta waves is approximately 25%; and the frequency proportion of the gamma waves is approximately 2%; wherein these frequency proportions can also be viewed as desired values of the frequency bands for this class. In a similar manner, in FIG. 2b the class of the EEG data is shown which is generated when the “dorsal attention network” of the brain is stimulated. FIGS. 2c through 2e show the frequency proportions of the class of EEG data given visual stimuli (FIG. 2c), given auditory stimuli (FIG. 2d), given sensory motor stimuli (FIG. 2e) and given stimuli which lead to a reaction of the medial prefrontal cortex (FIG. 2f).

The acquired MR data can now be divided corresponding to the classes defined with FIGS. 2a through 2f. For this, the frequency proportions are determined within the classical frequency bands (alpha, beta, gamma, delta, theta) of the EEG data or, respectively, EEG waves acquired simultaneously with the MR data in a time slice. A total amount is subsequently calculated for each of the six classes. The total amount of one of the six classes thereby corresponds to the sum of the absolute values of the differences from the determined frequency proportion of the acquired EEG data within the respective frequency band and the predefined desired value or, respectively, frequency proportion of the frequency band of the respective class. Six amount totals thereby exist. The MR data of the time slice are now associated with that class in which the amount total is smallest. This procedure corresponds to the previously described second variant of the preferred embodiment.

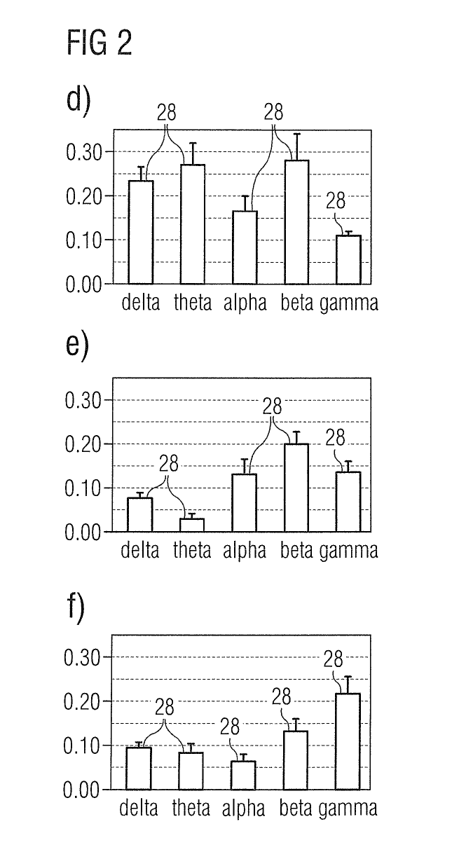

A different variant of a division of the MR data into various classes is shown in FIG. 3. In this variant, the frequency proportions within the classical frequency bands (alpha, beta, gamma, delta, theta) are also determined for each time slice s₁-s₁₀ for the EEG data acquired simultaneously with the MR data. The maximum among these five frequency proportions is determined. The class of the time slice then corresponds to the frequency class or the frequency band (alpha, beta, gamma, delta, theta) in which the maximum lies. This procedure corresponds to the first variant of the preferred embodiment that is described in the preceding. In this procedure, the EEG data (and therefore the MR data) are assigned to one of the five frequency classes in which the EEG data are predominantly situated during the time slice.

In the example shown in FIG. 3, the MR data 35 are acquired in ten time slices s₁ through s₁₀. Using the simultaneously acquired EEG data 26, the first three time slices s₁ through s₃ and the last two time slices s₉ and s₁₀ are subdivided into a class MR₁ (alpha); the fourth and fifth time slices s₄, s₅ are subdivided into a second class MR₂ (gamma); and the sixth through eighth time slice s₆ through s₈ are subdivided into a third class MR₃ (delta).

The evaluation of the MR data can now take place depending on the respective class MR₁ through MR₃, such that the evaluation of the MR data of the one class takes place in a different manner than the evaluation of the MR data of another class.

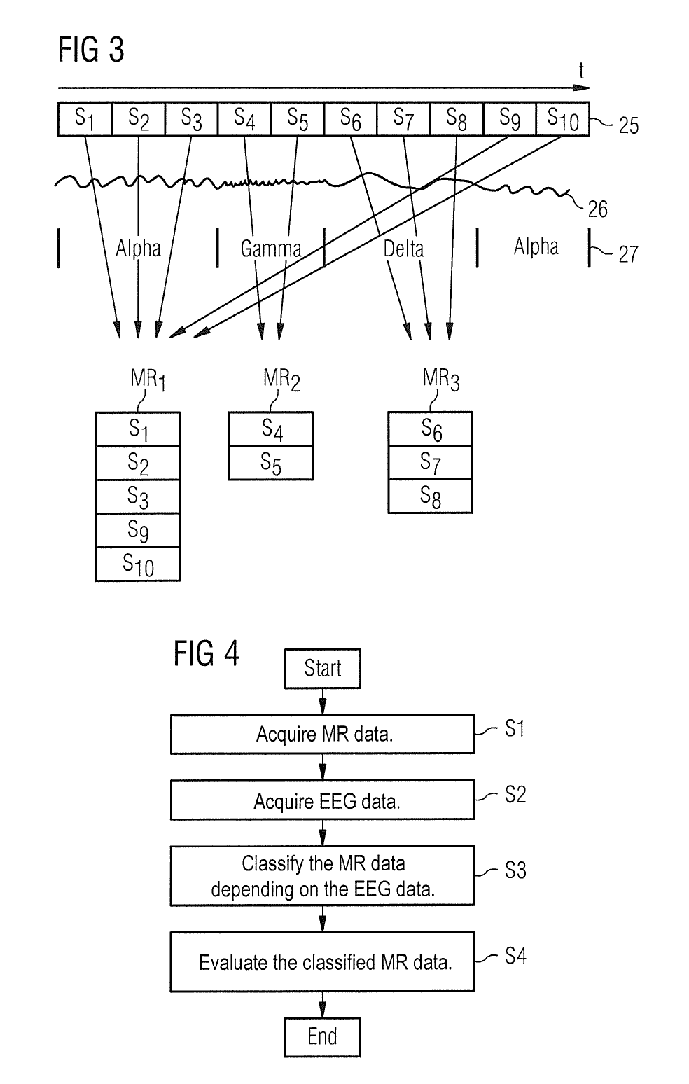

A workflow plan of a method according to the invention is presented in FIG. 4.

The MR data are acquired in a first Step S1, an the EEG data are acquired in the second step S2. Steps S1 and S2 are implemented simultaneously, such that the MR data and the EEG data of the examination subject are acquired simultaneously.

The MR data acquired simultaneously with these EEG data are classified (S3) under consideration of the EEG data, which means that the MR data are divided in particular into different classes depending on the EEG data. The classified MR data are evaluated (S4) depending on the respective class.

Although modifications and changes may be suggested by those skilled in the art, it is the intention of the inventor to embody within the patent warranted hereon all changes and modifications as reasonably and properly come within the scope of their contribution to the art.

We claim as our invention:

1. A method for functional magnetic resonance (MR) imaging of a predetermined volume segment of the brain of a living examination subject, comprising:
operating a magnetic resonance data acquisition unit to acquire MR data from a predetermined volume segment of the brain of a living examination subject;
simultaneously with acquisition of said MR data, acquiring EEG data from the examination subject; and
providing said MR data and said EEG data to a computerized processor and, in said processor, automatically evaluating said MR data dependent on said EEG data in order to produce an evaluation result indicative of brain activity of the examination subject, and making said evaluation result available in electronic form at an output of said processor.

2. A method as claimed in claim 1 comprising:
in said processor, implementing a spectral analysis of said EEG data; and
evaluating said MR data dependent on said spectral analysis.

3. A method as claimed in claim 1 comprising acquiring said MR data and said EEG data simultaneously in multiple, successive time slices;
in said processor, for each of said time slices, automatically determining a frequency spectrum of the EEG data acquired during the respective time slice, and determining a class for the respective time slice dependent on the frequency spectrum determined for the respective time slice;
in said processor, associating MR data acquired during the respective time slice with the class determined for the respective time slice; and
in said processor, evaluating MR data of a predetermined class differently than MR data in classes other than said predetermined class.

4. A method as claimed in claim 3 comprising:
in said processor, for each of said time slices, dividing an entirety of the frequency spectrum determined for a respective slice into a predetermined number of frequency bands;
designating a respective class to each of said frequency bands, so that a number of said classes equals a number of said frequency bands; and
assigning a class to MR data acquired during a respective time slice by assigning a class, among said number of classes, to the MR data acquired during the respective time slice that corresponds to a frequency band, among said number of frequency bands, in which the EEG data of the respective time slice are predominantly situated.

5. A method as claimed in claim 4 wherein one of said number of classes is the alpha wave frequency class, and using said alpha frequency wave class as said predetermined class and evaluating only MR data in said alpha wave frequency class to obtain said evaluation result.

6. A method as claimed in claim 3 comprising:
in said processor, dividing an entirety of said frequency spectrum of the EEG data into a predetermined number of frequency bands;
in said processor, defining a predetermined number of classes that are respectively defined as frequency proportions of said EEG data with respect to said frequency bands; and
for each of said time slices, determining the class thereof, from among said predetermined classes, that is a class having defined frequency proportions to which the EEG data acquired during the respective time slice best correspond.

7. A method as claimed in claim 1 comprising evaluating said MR data to produce, as said evaluation result, an MR image reconstructed from said MR data in which active brain centers are visually identifiable.

8. A method as claimed in claim 1 comprising:
acquiring said MR data and said EEG data in multiple, successive time slices;
in said processor, during each of said time slices, determining a frequency spectrum of the EEG data being acquired during the respective time slice;
evaluating MR data acquired in a respective time interval only when the frequency spectrum of the EEG data acquired in the respective time interval lies predominately in said predetermined frequency band; and
automatically ending acquisition of said MR data and said EEG data when a sum of time intervals, in which the frequency spectrum of the EEG data thereof is predominately in the predetermined frequency band, exceeds a predetermined time value.

9. A method as claimed in claim 1 comprising:
in said processor, automatically determining a frequency spectrum of said EEG data; and
from said processor, emitting humanly-perceptible user information dependent on said frequency spectrum.

10. A method as claimed in claim 1 comprising:
acquiring said MR data and said EEG data in multiple, successive time slices;
for each time slice, lowpass-filtering the EEG data thereof; and
in said processor, discarding MR data, from the evaluation of said MR data, acquired during any of said time slices in which a proportion of the lowpass-filtered EEG data, with respect to an entirety of the EEG data, is above a predetermined proportionality threshold.

11. A magnetic resonance (MR) apparatus for functional MR imaging, comprising:
an MR data acquisition unit;
a control unit configured to operate said MR data acquisition unit to acquire MR data from a predetermined volume segment of the brain of a living examination subject;
an electroencephalograph adapted for connection to said examination subject in said MR data acquisition unit;
said control unit being configured to operate said electroencephalograph to acquire EEG data from said examination subject simultaneously with acquisition of said MR data; and
a processor supplied with said MR data and said EEG data and configured to evaluate said MR data dependent on said EEG data to produce an evaluation result indicative of brain activity of the examination subject, said processor being configured to make said evaluation result available at an output of the processor in electronic form.

12. A non-transitory, computer-readable data storage medium encoded with programming instructions, said data storage medium being loaded into a computerized control and evaluation system of a magnetic resonance apparatus that also comprises an MR data acquisition unit and an electroencephalograph, said programming instructions causing said computerized control and evaluation system to:
operate said MR data acquisition unit to acquire MR data from the brain of a living examination subject;
operate the electroencephalograph to acquire EEG data from the examination subject simultaneously with acquisition of said MR data; and
evaluate the MR data dependent on the EEG data to produce an evaluation result indicative of brain activity of the examination subject, and make said evaluation result available in electronic form at an output of the control and evaluation system.
